(19) United States
(12) Patent Application Publication
(10) Pub. No.: US 2012/0065537 A1
(43) Pub. Date: Mar. 15, 2012

(54) SYSTEM FOR IN VIVO DETECTION OF A FUNCTIONAL AREA OF THE HUMAN BRAIN LEADING TO A HEMISPATIAL NEGLECT SYNDROME IN CASE OF LESION

(75) Inventors: Bastien Oliviero, Vincennes (FR); Michel Thiebaut De Schotten, Paris (FR); Paolo Bartolomeo, Paris (FR); Serge Kinkingnehun, Vitry Sur Seine (FR)

(73) Assignee: E(YE)BRAIN, Ivry Sur Seine (FR)

(21) Appl. No.: 13/321,876

(22) PCT Filed: May 25, 2010
(86) PCT No.: PCT/FR2010/051008

§ 371 Date: Nov. 22, 2011

(30) Foreign Application Priority Data
May 26, 2009 (FR) 0953463

(51) Int. Cl. A61B 5/0476

(52) U.S. Cl. 600/544

(57) ABSTRACT

A system for detecting a functional area (102) in the brain (104) of a person that causes hemispatial neglect in the person in the event of a lesion, includes: at least one electrode (106) for inducing a stimulus at a position of the brain (104) of the person; elements (108) for displaying a geometrical shape (112) after the stimulus; selection elements (110) for enabling the person to designate, according to predetermined instructions, a position relative to the geometrical shape (112); elements (108) for detecting the designated position; elements (116) for comparing the designated position with a position calculated according to the instructions; and elements (118, 120) for transmitting a signal on the basis of the comparison.

The invention concerns a system for in vivo detection of a functional area of the human brain leading to a hemispatial neglect syndrome in case of lesion. It also concerns a method for cerebral mapping of such an area.

The field of the invention is the field of:
the detection of a functional area causing a hemispatial neglect syndrome in case of lesion.
the mapping of the functional areas of the brain, more particularly those concerning the field of hemispatial neglect.

The invention thus provides practitioners with more precise knowledge of the functional areas of the brain of a patient, for example before operating on that patient.

Hemispatial neglect designates a difficulty in detecting, in identifying or in orienting oneself towards stimuli situated in the half of space which is contralateral to the injured hemisphere. In concrete terms, the persons affected have highly disabling behavior which may appear strange, for example neglecting all external things presented on the left, eating only half of what is on their plate, knocking the left shoulder when going through a door, only shaving or applying make-up to half the face, as if the left space did not exist.

Hemispatial neglect is caused by a cerebral lesion.

The presence of a cerebral tumor in a patient may sometimes require surgical ablation. In this specific case, it is essential to remove the part of the brain having the tumor without damaging the peripheral functional areas and thereby avoid post-operative hemispatial neglect syndrome in the patient.

At present, a functional area of the brain is detected in the following manner: the patient is woken during the operation. Batteries of tests for assessing hemispatial neglect are offered to him while the areas of his brain are disturbed by an electrical stimulation.

One of the best known batteries is the Battery for Hemispatial Neglect Assessment (BUHA), composed of several pencil-and-paper examinations. Among these tests are conventional tests, such as the bells consisting in circling the as many as possible of the bells presented on a sheet among other distracting items, the crossing out of lines consisting in crossing out all the lines presented on a sheet without distracting items. In all cases, “neglectful” patients neglect the target items presented in their left hemispace.

However, as indicated above these tests are carried out using a pencil and paper, and there is presently no system enabling these tests to be carried out more quickly and more efficiently, not only for the surgeon but also for the patient, especially if it is taken into account that these tests are carried out during an operation with the skull open.

Furthermore, these tests do not enable automatic and precise detection to be made of the functional area. Such detection is of capital importance since it enables almost the entirety of the tumor to be ablated, without disabling the patient by the destruction of a functional part of the brain.

It is also very important to perform such detection in the shortest possible time and to be able to provide a tool enabling the surgeon to be assisted during the operation or to prepare that operation.

An object of the invention is to mitigate these drawbacks.

Another object of the invention is to provide an automated system for detecting a functional area of a person's brain that leads to hemispatial neglect in said person in case of lesion.

Lastly, another object of the invention is to provide a system performing more effective detection of a functional area of a person's brain, which is practical to use during an operation and which presents less danger for the patient.

The invention enables the aforementioned objects to be attained by a system for detecting a functional area of a person's brain leading in said person to hemispatial neglect in case of lesion, said system comprising:
at least one electrode adapted to induce a stimulus in a position of the brain of said person,
means for displaying a geometric shape further to/during said stimulus,
selection means enabling said person to point, according to a predetermined instruction, to a position relative to said geometric shape,
means for detecting the position pointed to,
means for comparing said pointed to position to a position computed according to said instruction, and
means for emitting a signal on the basis of said comparison.

The system according to the invention makes it possible perform automated detection of a functional area of the brain.

The detection of a functional area by the system according to the invention is more practical and safer than a detection of the state of the art. To be precise, with the system according to the invention the operator may focus his attention fully on the patient's brain and on each of the electrode positions in the brain since a signal will inform the operator of the results by the application of the instruction by the person concerned throughout the test.

Furthermore, the detection of a functional area of the brain by the system according to the invention is faster, since the means used are electronic and/or computer means enabling the person and the operator to perform the detection without interruption.

Furthermore, the system for detecting a functional area according to the invention may be used directly in an operating theater with immediate access to the results, or in advance of an operation on a patient.

In a particular embodiment, the means for detecting the position pointed to may comprise a touch screen, said touch screen also displaying said geometric shape.

The touch screen may be used with a pointer such as a stylus or directly with the finger.

Thus, the display means and the selection means comprise a touch screen.

In a particularly advantageous version, the means for emitting a signal are adapted to emit a different signal for each of the following cases:
the distance between the position pointed to and the computed position is less than a predetermined distance,
the distance between the position pointed to and the computed position is greater than said predetermined distance but less than twice said distance,
the distance between the position pointed to and the computed position is greater than twice said predetermined distance.

Thus, depending on the signal emitted, the operator can directly know whether the simulated position is located in a functional area of the brain or not.

The system further comprises means for determining the position of the stimulus. These means may comprise a camera filming a scene and thus the position of each of the stimuli.

The system according to the invention may further comprise means for storing the result of the comparison in association with the position of the stimulus, for a plurality of different positions.

The storage means may be incorporated locally or be located in a remote database. In the latter case, the system may further comprise means for connecting to the remote database, it being possible for those means for connecting to comprise wireless and/or wired means for connecting via an existing network for example.

Advantageously, the system according to the invention may further comprise means for mapping the functional area on the basis of the position of each of the stimuli and the result of the comparison for said position, based on a map of the brain of the person, obtained by medical imaging means known to the person skilled in the art, such as magnetic resonance imaging (MRI). Thus a map in 2 or 3 dimensions of the functional area in the brain may be produced.

The map produced may be used during the surgical operation on a brain or before the operation to prepare for the ablation of the brain tumor.

Such a map of the brain showing the functional area makes it possible to better visualize the functional area in the brain.

The mapping means may comprise one or more computer programs.

According to a particularly advantageous version, the means for emitting a signal comprise means for emitting an acoustic signal. The acoustic signal may have different frequency, duration or iterations depending on the result of the comparison.

An acoustic signal has the advantage for the operator of not having to direct his visual attention away from the person's brain to know the result of the comparison, which reduces the risk of mishandling by the operator which could have major consequences for the person's health.

The means for emitting a signal may comprise means for emitting a visual signal.

Such a visual signal may furthermore be used in addition to an acoustic signal.

According to another aspect of the invention, there is provided a method of mapping a functional area of a person's brain leading to a hemispatial neglect syndrome in that person in case of lesion, said method comprising the following steps:
generating stimuli in the brain using at least one electrode in a plurality of positions,
for each of said stimuli, detecting the position of said stimuli relative to a predefined reference,
determining, for each of the stimuli, a result obtained by the person in a test for assessing hemispatial neglect,
determining the limits of said area on the basis of the results obtained for the all the stimuli, and
displaying said area.

Advantageously, the position of each stimulus may be determined in three dimensions. Thus, the position of each stimulus comprises coordinates in three dimensions relative to a predefined reference. Thus, the detection of the limits of the functional area may be carried out in three dimensions.

The detection of the limits of the functional area in three dimensions may be carried out by virtue of generating stimuli on the surface of the brain, at positions situated on the outer surface of the brain and situated in surface areas of the brain revealed for example by the ablation of a tumor, which corresponds to positions within the thickness of the brain before ablation.

Such detection enables three dimensional mapping which can be used for viewing the functional area before or during the operation.

Furthermore, such a map in three dimensions may be used by a digital apparatus for assisting the surgery used during the ablation of the area of the brain tumor.

Other advantages and characteristics of the invention will appear on studying the detailed description of an embodiment which is in no way limiting, and of the accompanying drawings in which:

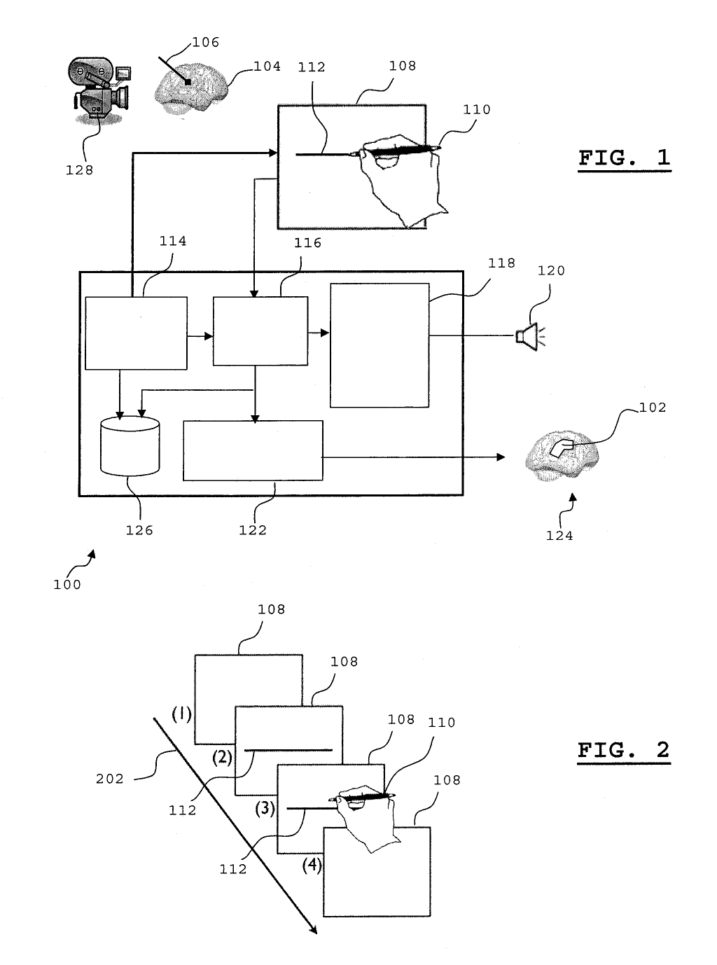

FIG. 1 is a diagram of a system according to the invention; and

FIG. 2 is an example of a test used by the system of FIG. 1 for determining a functional area of the brain of a person suffering from hemispatial neglect.

FIG. 1 is a diagram of a system 100 for detecting a functional area 102 of the brain 104 of a patient, the lesion of which causes a hemispatial neglect syndrome.

The system 100 comprises:
at least one electrode 106 adapted to induce a stimulus in a position of the brain 104 of the patient, Each stimulus is an electrical signal of very low voltage and of a few mA on the patient's brain 104;
a touch screen 108 on which are displayed one or more geometric shapes during or just after the stimulus;
a stylus 110 enabling the patient to point to a position on the touch screen 108 in accordance with a predetermined instruction. The position to point to is requested in relation to the geometric shape. In the example represented in FIG. 1 the geometric shape is a line segment 112 and the instruction is to point to the middle of the line segment 112 with the stylus 110;
a module 114 for generating the geometric shape on the display screen 108. This module 114 for generating the shape determines the display position of the line segment 114 on the display screen 108, as well as the instruction, in particular the position which must be pointed to by the patient, i.e. the middle of the line segment 112.

The position pointed to by the patient with the stylus 110 is detected by the touch screen 108 and communicated to a comparison module 116. This comparison module 116 receives the position pointed to by the patient from the touch screen 108 and, from the stimuli generation module 114, the exact position which the patient should point to according to the instruction. The comparison module 116 compares the two positions and determines, on the basis of one or more predetermined thresholds, the level of accuracy of the response given by the patient.

The level of accuracy is communicated to the module 118 for generating an acoustic signal. This module 118 generates a different acoustic signal according to the level of accuracy. The acoustic signal associated with the level of accuracy communicated by the comparison module 116 is emitted by a loudspeaker 120 connected to the acoustic signal generating module 118.

The level of accuracy as well as the position of the electrode 106 are also communicated to a mapping module 122. According to the level of accuracy for multiple positions of stimulus on the brain 104, the mapping module 122 generates a map 124 of the brain 104 in three dimensions on which the functional area 102 of the brain 104 is represented, here the area which when simulated induces hemispatial neglect.

The generation of the map 124 of the brain 104 in three dimensions by the generating module 122 may be carried out either in real time during the test of the patient with the stimuli or offset in time after the test.

The map generated 124 may be displayed on a display screen not shown in FIG. 1.

All or some of the data as well as the display map may be stored in a database 126.

The system according to the invention may furthermore comprise means for converting the map 124 into a predetermined language to enable the map 124 to be used by a digital apparatus for aiding the surgeon during the ablation of the brain tumor area.

The system may furthermore comprise a camera 128 filming the actions of the operating surgeon and recording the acoustic signals emitted. The data recorded by the camera 128 may be used by the generating module 122 for generating the map 124 of the brain in three dimensions showing the functional area of the patient's brain.

FIG. 2 is a representation of an example of a test for assessing hemispatial neglect, used by the system of FIG. 1 to determine a functional area of the brain.

This is a computerized cognitive test of line segment bisection using the touch screen 108. An high-pitched acoustic signal accompanies the appearance of the horizontal line of 20 cm. The instruction given to the patient is to point to the center of the line segment displayed on the touch screen 108. The subject must therefore mark the center of the line segment with the stylus.

A low-pitched acoustic error signal indicates if the subject is more than 6.5 mm (predetermined distance) from the center (single beep) or more than 13 mm from the center (double beep). There is no low-pitched acoustic signal if the subject marks the line precisely in the center (less than 6.5 mm of error). The thresholds of 6.5 mm (predetermined distance) and of 13 mm (twice the predetermined distance) are not arbitrary but correspond to thresholds defined in advance in the literature as being statistically abnormal.

This test may in particular be used in a pre-operative context, so as not to disable the patient during a neurosurgical operation.

In a particular embodiment, an example of a system tested comprises a portable computer with a touch screen and stylus for response, with the Windows XP operating system.

A computer program performing the functions described above loops until the “escape” key is pressed.

A plurality of stimuli is progressively displayed. Axis 202 in FIG. 2 symbolizes time and the numbers in brackets in FIG. 2 symbolize the order of stimuli displayed on the touch screen 108 as the test progresses. The display duration of the different stimuli is different. According to a particular example:
the stimulus (1) comprises two scenes, a first of 1000 ms, then an acoustic signal, then a second scene of 500 ms followed by an acoustic signal,
the stimulus (2) is a scene of 5000 ms,
the stimulus (3) no scene is displayed, and
the stimulus (4) is an acoustic signal followed by a scene of 500 ms.

For each test the computer program records the response in a text file and emits an acoustic signal according to the reply.

The recorded response is constituted by the response time from the appearance of the line, the position in pixels of the point marked by the subject, the distance in millimeters of that point relative to the center of the line, the percentage of deviation and that side of the deviation.

If the subject does not respond at the end of 5 seconds, an acoustic error signal is emitted and the program records in the response file that there was no response to that test.

Naturally, the invention is not limited to the examples which have just been described and numerous modifications may be made to these examples without departing from the scope of the invention.

1. A system for detecting a functional area (102) of a person's brain (104) leading in said person to hemispatial neglect in case of lesion, said system comprising:
at least one electrode (106) adapted to induce a stimulus in a position of the brain (104) of said person,
means (108) for displaying a geometric shape (112) further to said stimulus,
selection means (110) enabling said person to point, according to a predetermined instruction, to a position relative to said geometric shape (112),
means for detecting (108) the position pointed to,
means (116) for comparing said pointed to position to a position computed according to said instruction, and
means (118,120) for emitting a signal on the basis of said comparison.

2. A system according to claim 1, characterized in that the means for detecting the position pointed to comprise a touch screen (108), said touch screen (108) also displaying said geometric shape (112).

3. A system according to claim 1, characterized in that the means (118,120) for emitting a signal are adapted to emit a different signal for each of the following cases:
the distance between the position pointed to and the computed position is less than a predetermined distance,
the distance between the position pointed to and the computed position is greater than said predetermined distance but less than twice said distance,
the distance between the position pointed to and the computed position is greater than twice said predetermined distance.

4. A system according to claim 1, characterized in that it further comprises means (114) for determining the position of the stimulus.

5. A system according to claim 1, characterized in that it further comprises means (126) for storing the result of the comparison in association with a position of the stimulus, for a plurality of different positions.

6. A system according to claim 5, characterized in that it further comprises means (122) for mapping the functional area (102) on the basis of the position of each of the stimuli and the result of the comparison for said position.

7. A system according to claim 1, characterized in that the means for emitting a signal comprise means (118,120) for emitting an acoustic signal.

8. A system according to claim 1, characterized in that the means for emitting a signal comprise means for emitting a visual signal.

9. A method of mapping a functional area (102) of a person's brain (104) leading to a hemispatial neglect syndrome in that person in case of lesion, said method comprising the following steps:
generating stimuli in the brain of said person using at least one electrode (106) in a plurality of positions,
for each of said stimuli, detecting the position of said stimuli relative to a predefined reference,
determining, for each of the stimuli, a result obtained by the person in a test for assessing hemispatial neglect,
determining the limits of said area on the basis of the results obtained for the all the stimuli, and
displaying said area.

10. A method according to claim 9, characterized in that The position of each stimulus comprises coordinates in three dimensions, the detection of the limits of the functional area (102) being carried out in three dimensions.

11. A system according to claim 2, characterized in that the means (118,120) for emitting a signal are adapted to emit a different signal for each of the following cases:
the distance between the position pointed to and the computed position is less than a predetermined distance,
the distance between the position pointed to and the computed position is greater than said predetermined distance but less than twice said distance,
the distance between the position pointed to and the computed position is greater than twice said predetermined distance.

12. A system according to claim 2, characterized in that it further comprises means (114) for determining the position of the stimulus.

13. A system according to claim 2, characterized in that it further comprises means (126) for storing the result of the comparison in association with a position of the stimulus, for a plurality of different positions.
